(19) United States
(12) Patent Application Publication
(10) Pub. No.: US 2021/0275691 A1
(43) Pub. Date: Sep. 9, 2021

(54) SCREENING ASSAY TO ASSESS TOPICAL DELIVERY OF BIOLOGICAL THERAPEUTIC AGENTS

(71) Applicant: Allergan, Inc., Irvine, CA (US)

(72) Inventors: Jie Shen, Irvine, CA (US); Hsiang Ho, Irvine, CA (US); Lydia Andrew-Jones, Lake Forest, CA (US); Betsy Bloom, Anaheim, CA (US); Greg Nicholson, Cypress, CA (US); Lance Steward, Irvine, CA (US); Amy Brideau-Anderson, San Clemente, CA (US); Terrence Hunt, Temecula, CA (US); Ron Broide, San Marcos, CA (US); David Rupp, San Pedro, CA (US); David Canty, Irvine, CA (US)

(21) Appl. No.: 17/058,037

(22) PCT Filed: May 30, 2019
(86) PCT No.: PCT/US2019/034664

§ 371 Date: Nov. 23, 2020

Related U.S. Application Data
(60) Provisional application No. 62/677,800, filed on May 30, 2018.

(51) Int. Cl. A61K 49/00; G01N 33/50; A61K 9/00

(52) U.S. Cl. CPC A61K 49/0008; C12Y 304/24069; A61K 9/0014; G01N 33/5082

(57) ABSTRACT

An in vivo assay to evaluate topical delivery of a biologic agent from a formulation is provided. The assay comprises treating a skin area of a CD hairless rat with tape stripping to disrupt the stratum corneum in the skin area to define a treated skin area and topically applying a formulation to the treated skin area. The formulation, in one embodiment, comprises the biologic agent and a pharmaceutically acceptable carrier. Delivery of the biologic agent into the skin from the topically applied formulation is evaluated via in vitro or in vivo techniques.

### CROSS-REFERENCES TO RELATED APPLICATIONS

This application claims the benefit of U.S. Provisional Application No. 62/677,800, filed May 30, 2018, which is incorporated herein by reference in its entirety.

### TECHNICAL FIELD

The present disclosure relates to a screening assay to analyze delivery of a biologic agent to the dermis, hypodermis and/or superficial muscles from a topically applied formulation.

### BACKGROUND

Human skin is a readily accessible surface for delivery of beneficial agents. Skin of an average adult body covers a surface of approximately 2 m², and receives about one-third of the blood circulating through the body. Skin contains an uppermost layer, epidermis which has morphologically distinct regions; basal layer, spiny layer, stratum granulosum and the upper most stratum corneum. For topical delivery of a beneficial agent to the skin and for transdermal delivery of a beneficial agent to the system, the agent must overcome the barrier properties of the stratum corneum. The stratum corneum is selectively permeable to agents placed on it, and allows only relatively lipophilic compounds with a molecular weight below 400 Daltons to pass across it. A method to evaluate delivery of a beneficial agent from a topically applied formulation would be useful. In particular, a method that models human skin is desirable to assess delivery of beneficial agents into skin, for delivery to a human.

### BRIEF SUMMARY

In one aspect, an in vivo assay to evaluate topical delivery of a biologic agent from a formulation is provided. The assay comprises treating a skin area of a CD hairless rat with tape stripping to disrupt the stratum corneum in the skin area to define a treated skin area; topically applying a formulation to the treated skin area, the formulation comprising the biologic agent and a pharmaceutically acceptable carrier; and evaluating delivery of the biologic agent into the skin from the topically applied formulation.

In one embodiment, treating comprises applying and removing a strip of tape between 2-9 times. In other embodiments, treating comprises applying and removing a strip of tape between 3-7 times. In still other embodiments, treating comprises applying and removing a strip of tape between 4-6 times.

In yet another embodiment, treating comprises applying and removing a strip of tape using a different tape strip for each step of applying.

In still another embodiment, the step of applying comprises placing the tape strip to the skin applying mild thumb pressure for about 5 seconds.

In one embodiment, the step of removing comprises removing in a single direction (unidirectional). In another embodiment, the step of removing comprises removing unidirectionally at an angle between about 35-90°.

In other embodiments, treating comprises treating with tape stripping using a strip of tape with a synthetic adhesive, such as a selected from an acrylic adhesive, a silicone adhesive and a polyurethane adhesive. In one embodiment, the synthetic adhesive is free of rubber and/or latex. In one embodiment, the synthetic adhesive is a hypoallergenic acrylic adhesive. Exemplary adhesive tapes include those sold under the brand names D-SQUAME®, Corneofix®, Blenderm™, and 3M-Scotch 845 Book Tape.

In one embodiment, evaluating comprises evaluating in vitro.

In one embodiment, in vitro evaluating comprises a histologic evaluation of the treated skin area.

In another embodiment, evaluating comprises evaluating in vivo.

In still other embodiments, the biologic agent is optically labeled and said evaluating comprises evaluating the treated skin area for the optical label.

In an embodiment, evaluating for the optical label is via microscopy or spectroscopy.

In another embodiment, evaluating comprises evaluating using a functional assay of the rat.

In another embodiment, evaluating comprises a determining a concentration of the biologic agent or a metabolite thereof in the blood of the rat.

In one embodiment, the assay further comprises comparatively evaluating delivery of the biologic agent from the formulation applied to CD hairless rat skin not treated with tape stripping.

The step of topically applying, in one embodiment, comprises topically applying a formulation comprising topically applying a formulation with a biologic agent having a molecular weight of greater than about 40,000 Daltons.

In one embodiment, topically applying comprises topically applying a formulation comprising a Clostridial derivative. In an embodiment, the Clostridial derivative is a botulinum toxin.

In another aspect, a method to evaluate skin penetration of a biologic agent from a topically applied formulation is provided. The method comprises removing via tape stripping stratum corneum from a skin area of a CD hairless rat to define a conditioned skin area; applying topically a formulation to the conditioned skin area, the formulation comprising a biologic agent and a pharmaceutically acceptable carrier; and evaluating penetration of the biologic agent into the skin.

In another aspect, an in vivo assay to evaluate depth of penetration of a topically applied biologic agent, is provided. The assay comprises treating a skin area on a leg of a CD hairless rat by applying and removing adhesive tape strips to disrupt the stratum corneum in the skin with no disruption to underlying epidermis to define a conditioned skin area; topically applying a formulation to the conditioned skin area, the formulation comprising a biologic agent with a molecular weight of at least about 150,000 Daltons and a pharmaceutically acceptable carrier; and evaluating depth of penetration of the biologic agent into dermis or into a superficial muscle from the topically applied formulation.

In one embodiment, the skin area on the leg of the test animal is an areas of skin in the region of the tibialis anterior muscle. In another embodiment, the evaluating depth of penetration of the biologic agent comprises evaluating using a functional assay of the tibialis anterior muscle. In one embodiment, the functional assay is a rodent motor assay, such as the rat digit abduction score. In another embodiment, the evaluating depth of penetration of the biologic agent comprises evaluating images of tibialis anterior muscle following immunohistochemical staining for SNAP25₁₉₇ in the pre-syntaptic motor nerve terminal and motor nerve axons using a recombinant monoclonal antibody. In another embodiment, evaluating the depth of penentration of the biologic agent comprises or additionally comprises evaluating images of tibialis anterior muscle samples for the presence of total neuromuscular junctions using fluorescent-labeled alpha-bungarotoxin.

### BRIEF DESCRIPTION OF THE DRAWINGS

The following drawings are presented to illustrate aspects and features of embodiments of the present screening assay and methods.

FIGS. 1A-1C are histologic images of rat skin from the leg of a CD hairless rat before (FIG. 1A) and after treating by tape stripping by applying and removing a new strip of tape 5 times (FIG. 1B) or 10 times (FIG. 1C), all images are 200× magnification, the upper arrow in each image indicates the stratum corneum (or location of stratum corneum that has been removed in FIG. 1C image), and the lower arrow indicates the epidermis.

FIGS. 2A-2C are histologic images of tibialis anterior muscle in the CD hairless rat following immunohistochemcial staining for the presence of SNAP25₁₉₇ in the pre-synaptic motor nerve terminals (MNT) and motor nerve (MN) axons using a recombinant monoclonal antibody (FIG. 2A) and for the presence of total neuromuscular junctions (NMJs) using fluorescent-labeled α-bungarotoxin (α-Bgt), which binds to post-synaptic nicotinic acetylcholine receptors (nAChR) (FIG. 2B) after no tape stripping before topical application of a formulation comprising a botulinum toxin, where FIG. 2C merges the images of FIGS. 2A-2B.

FIGS. 2D-2F are histologic images of tibialis anterior muscle in the CD hairless rat following immunohistochemcial staining for the presence of SNAP25₁₉₇ in the pre-synaptic motor nerve terminals (MNT) and motor nerve (MN) axons using a recombinant monoclonal antibody (FIG. 2D) and for the presence of total neuromuscular junctions (NMJs) using fluorescent-labeled α-bungarotoxin (α-Bgt), which binds to post-synaptic nicotinic acetylcholine receptors (nAChR) (FIG. 2E) after tape stripping five times before topical application of a formulation comprising a botulinum toxin, where FIG. 2F merges the images of FIGS. 2D-2E.

### DETAILED DESCRIPTION

### Definitions

The following definitions apply herein:

“About” or “approximately” as used herein means within an acceptable error range for the particular value as determined by one of ordinary skill in the art, which will depend in part on how the value is measured or determined, (i.e., the limitations of the measurement system). For example, “about” can mean within 1 or more than 1 standard deviations, per practice in the art. Where particular values are described in the application and claims, unless otherwise stated, the term “about” means within an acceptable error range for the particular value.

“Administration” or “to administer” means the step of giving (i.e. administering) a botulinum toxin to a subject, or alternatively a subject receiving a pharmaceutical composition.

“Botulinum toxin” means a neurotoxin produced by Clostridium botulinum, as well as a botulinum toxin (or the light chain or the heavy chain thereof) made recombinantly by a non-Clostridial species. The term “botulinum toxin”, as used herein, encompasses Botulinum toxin serotype A (BoNT/A), Botulinum toxin serotype B (BoNT/B), Botulinum toxin serotype C (BoNT/C), Botulinum toxin serotype D (BoNT/D), Botulinum toxin serotype E (BoNT/E), Botulinum toxin serotype F (BoNT/F), Botulinum toxin serotype G (BoNT/G), Botulinum toxin serotype H (BoNT/H), Botulinum toxin serotype X (BoNT/X), and mosaic Botulinum toxins and/or subtypes and variants thereof. “Botulinum toxin”, as used herein, also encompasses a “modified botulinum toxin”. Further “botulinum toxin” as used herein also encompasses a botulinum toxin complex, (for example, the 300, 600 and 900 kDa complexes), as well as the neurotoxic component of the botulinum toxin (150 kDa) that is unassociated with the complex proteins.

“Clostridial derivative” refers to a molecule which contains any part of a clostridial toxin. As used herein, the term “clostridial derivative” encompasses native or recombinant neurotoxins, recombinant modified toxins, fragments thereof, a Targeted vesicular Exocytosis Modulator (TEM), or combinations thereof.

“Clostridial toxin” refers to any toxin produced by a Clostridial toxin strain that can execute the overall cellular mechanism whereby a Clostridial toxin intoxicates a cell and encompasses the binding of a Clostridial toxin to a low or high affinity Clostridial toxin receptor, the internalization of the toxin/receptor complex, the translocation of the Clostridial toxin light chain into the cytoplasm and the enzymatic modification of a Clostridial toxin substrate. Non-limiting examples of Clostridial toxins include a Botulinum toxin like BoNT/A, a BoNT/B, a BoNT/C₁, a BoNT/D, a BoNT/E, a BoNT/F, a BoNT/G, a Tetanus toxin (TeNT), a Baratii toxin (BaNT), and a Butyricum toxin (BuNT). The BoNT/C₂ cytotoxin and BoNT/C₃ cytotoxin, not being neurotoxins, are excluded from the term “Clostridial toxin.” A Clostridial toxin disclosed herein includes, without limitation, naturally occurring Clostridial toxin variants, such as, e.g., Clostridial toxin isoforms and Clostridial toxin subtypes; non-naturally occurring Clostridial toxin variants, such as, e.g., conservative Clostridial toxin variants, non-conservative Clostridial toxin variants, Clostridial toxin chimeric variants and active Clostridial toxin fragments thereof, or any combination thereof. A Clostridial toxin disclosed herein also includes a Clostridial toxin complex. As used herein, the term “Clostridial toxin complex” refers to a complex comprising a Clostridial toxin and non-toxin associated proteins (NAPs), such as, e.g., a Botulinum toxin complex, a Tetanus toxin complex, a Baratii toxin complex, and a Butyricum toxin complex. Non-limiting examples of Clostridial toxin complexes include those produced by a Clostridium botulinum, such as, e.g., a 900-kDa BoNT/A complex, a 600-kDa BoNT/A complex, a 300-kDa BoNT/A complex, a 500-kDa BoNT/B complex, a 500-kDa BoNT/C₁ complex, a 500-kDa BoNT/D complex, a 300-kDa BoNT/D complex, a 300-kDa BoNT/E complex, and a 300-kDa BoNT/F complex.

The term “intact skin” refers to skin that retains its natural barrier function, and has not been altered by chemical means or physical treatment in a way that may harm the barrier function of the stratum corneum. Conversely, “non-intact” skin refers to skin that has been treated in a way that harms the barrier function of stratum corneum.

“Local administration” means administration of a pharmaceutical agent at or to the vicinity of a site on or within an animal body, at which site a biological effect of the pharmaceutical is desired, such as via, for example, intramuscular or intra- or subdermal injection or topical administration. Local administration excludes systemic routes of administration, such as intravenous or oral administration. Topical administration is a type of local administration in which a pharmaceutical agent is applied to a patient's skin.

The term “molecular weight” refers to the sum of the atomic weights of all atoms constituting a molecule, and can be numerically expressed in Dalton (Da).

A “biologic agent” intends a molecule that is biologically active and has a molecular weight of about 5,000 Daltons or greater, or has a molecular weight in a range of values specified herein.

The term “passive transdermal delivery” refers to delivery of an active agent by placing it on the surface of skin whereby it permeates into the skin as a function of concentration gradient between the higher drug concentration on the skin surface and the lower drug concentration within the skin.

“TEMs”, an abbreviation for Targeted Exocytosis Modulators, are retargeted endopeptidases that direct the catalytic activity of the light chain to specific types of neuronal cells or to target cells that were not affected by botulinum toxins expanding the beneficial clinical effect of inhibition of exocytosis in several human diseases.

Topical application” or “topically applying”, as used herein, is meant directly laying or spreading upon epidermal tissue, especially outer skin, where the stratum corneum layer may be intact or non-intact (i.e., disrupted).

“Topical delivery” or “topical administration”, and the like, as used herein mean passage of a topically applied active agent into the skin for localized delivery to the skin.

“Transdermal” as used herein means passage into and/or through skin for localized delivery to superficial muscles or for systemic delivery of an active agent.

“Treating” or “treatment” means to alleviate (or to eliminate) at least one symptom (such as, for example, hip and groin pain), either temporarily or permanently.

“Therapeutically effective amount” refers to an amount sufficient to achieve a desired therapeutic effect.

“Variant” means a clostridial neurotoxin, such as wild-type botulinum toxin serotype A, B, C, D, E, F, G, H, X, mosaic Botulinum toxins and/or subtypes, hybrids, chimeras thereof that has been modified by the replacement, modification, addition or deletion of at least one amino acid relative to wild-type botulinum toxin, which is recognized by a target cell, internalized by the target cell, and catalytically cleaves a SNARE (SNAP (Soluble NSF Attachment Protein) Receptor) protein in the target cell.

### Screening Assay and Method of Use

For topically applied agents, the primary barrier for skin penetration is the stratum corneum. One approach to overcoming the barrier posed by the stratum corneum is to disrupt this layer, to permit a topically applied agent to partition into the skin layers beneath the stratum corneum. A screening assay that models human skin with a disrupted stratum corneum or wherein the thickness of the stratum corneum more closely resembles that of human skin would be beneficial to studies evaluating topical delivery of an agent and/or topical delivery of an agent from various formulations. The present assay and methods provide such a model, where stratum corneum is disrupted and/or thinned and the underlying epidermis remains intact and undamaged. The model uses CD hairless rats. The stratum corneum of these rats is often thicker than that on human skin, but the viable epidermal layers is often thinner than human skin. To reduce the thickness of the stratum corneum at the site of application in CD hairless rats, without damaging the underlying epidermis, a tape stripping technique was developed. Using the tape stripping technique stratum corneum was removed without damage to the underlying epidermis.

Example 1 describes a study where the effect of tape stripping on skin of CD hairless rats was evaluated. In the leg area of the animals, tape strips were applied and removed 5, 10, 20 or 30 times, using a new strip of tape each time. The effect of the tape stripping to reduce the thickness of the stratum corneum, without damaging the underlying epidermis, was evaluated by inspecting cross sections of the skin.

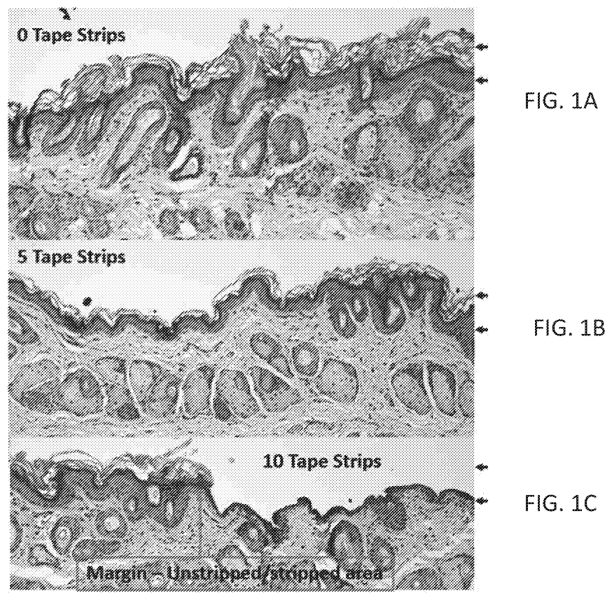

FIG. 1A shows a skin cross section from the leg region of a CD hairless rat that was not tape stripped. The stratum corneum is visible in the images, identified by the arrows in the right side of the image. FIG. 1B is an image of the leg skin area of a CD hairless rat treated by applying and removing a fresh strip of tape 5 times. Approximately ⅔ of the stratum corneum was removed from the surface of the skin, with no apparent damage to underlying epidermis. FIG. 1C shows an image of the leg skin area of a CD hairless rat treated by applying and removing a fresh strip of tape 10 times. The image shows that ten or more tape strips removed all the stratum corneum except in the isthmus of hair follicles, and caused damage to the underlying epidermis.

Accordingly, an in vivo assay to evaluate topical delivery of a biologic agent from a formulation is provided. The assay comprises treating a skin area of a CD hairless rat with tape stripping to disrupt the stratum corneum in the skin area to define a treated skin area before topically applying a formulation to the treated skin area. In one embodiment, the treating comprises applying and removing a strip of tape between 1-9 times, or between 1-8, 1-7, 1-6, 2-9, 2-8, 2-7, 2-6, 2-5, 2-4, 2-3, 3-9, 3-8, 3-7, 3-6, 3-5, 3-4, 4-9, 4-8, 4-7, 4-6, 4-5, 5-9, 5-8, 5-7, 5-6, 6-9, 6-8, 6-7, 7-9, 7-8, or 8-9 times. In one embodiment, each tape stripping is performed with a tape strip that is applied to the skin and removed, and the next tape stripping is performed with a new, different tape strip. In another embodiment, each step of applying is conducted with a new tape strip that has not been previously applied to skin and removed.

In another embodiment, each step of applying comprises placing the tape strip on the skin and applying pressure for a period of time. In one embodiment, applying pressure comprises pressing the tape onto the skin with a finger or a thumb. In one embodiment, finger or thumb pressure is applied to the tape strip gently, moderately or firmly. In other embodiments, applying pressure comprises applying pressure to the tape after its application on the skin using a roller or a stamp that apply a known, defined pressure, such as a pressure in the range of 1-10 Newtons, or 2-8 Newtons. The pressure can be applied for a period of less than about 30 seconds, less than about 20 seconds, less than about 15 seconds, or for between about 1-15 seconds, 1-12 seconds, 1-10 seconds, 1-8 seconds, 1-7 seconds, 1-6 seconds, 1-5 seconds, 2-15 seconds, 2-12 seconds, 2-10 seconds, 2-8 seconds, 2-7 seconds, 2-6 seconds, 2-5 seconds, 3-15 seconds, 3-12 seconds, 3-10 seconds, 3-8 seconds, 3-7 seconds, 3-6 seconds, 3-5 seconds, or for 1 second, 2 seconds, 3 seconds, 4 seconds, 5 seconds, 7 seconds, 8 seconds, 9 seconds, 10 seconds, 12 seconds, or 15 seconds.

In one embodiment, the step of removing the tape strip comprises removing the tape strip unidirectionally, either with a thumb and finger or with an instrument, such as forceps. In other embodiments, the unidirectional removal is done with the tape strip at an angle relative to the skin surface of between about 30-90°, about 40-85°, or about 45-80°.

In another embodiment, the treating is performed with a synthetic adhesive type, such as a polyacrylate ester adhesive tape available under the brand name D-SQUAME® tape strips (Clinical and Derm LL, formerly Cuderm® Corporation). In another embodiment, the treating is performed with a synthetic adhesive tape available under the brand name Corneofix® tape (Courage+Khazaka electronic GmbH, Cologne, Germany), or under the brand names Blenderm™ tape (3M Corporation), or 3M-Scotch 845 Book Tape.

The screening assay described herein was used to evaluate skin penetration of a biologic agent from a topically applied formulation. Botulinum toxin was used as a model biologic agent, and the toxin used had a molecular weight of about 150 kDa. In order to measure the skin penetration of the botulinum neurotoxin, a rodent motor assay known as the rat digit abduction score (DAS) was used. This functional assay is a physiological assay that is used to determine the efficacy of BoNT/A on local muscle weakening (Broide, R. S. et al., Toxicon, 71:18-24 (2013)). Following intramuscular (IM) injection, the toxin elicits a dose-dependent reduction in the animal's ability to produce a characteristic hind limb startle response and the degree of this response is scored on a five-point scale. Additionally, the presence of functional BoNT/A in motor nerves within the muscle can be validated by immunohistochemical (IHC) staining for the BoNT/A-cleaved SNAP25 substrate (SNAP25₁₉₇) using a highly selective antibody (Rheaume, C. et al., Toxins (Basel), 7(7), 2354-2370 (2015); Cai, B. B. et al., Neuroscience, 352:155-169 (2017)). The rat DAS assay generally involves IM injection of BoNT/A into one of the hindlimb calf muscles, such as the tibialis anterior (TA) followed by DAS scoring.

In the study of Example 2, treatment of CD hairless rats with tape strips was performed to disrupt the stratum corneum to determine whether this action can facilitate the delivery of functional botulinum toxin, BoNT/A, from the skin surface to the underlying muscle. Based on the data from Example 1, the skin area overlying the TA muscle was conditioned by applying and removing tape strips five times. The tape was applied to the skin using a tool (forceps) and was pressed for about 5 seconds. The tape was removed from the skin unidirectionally. The process was repeated for a total number of five strips. A control group received no tape stripping. Then, in both the control group and the skin treated group, 150 kDa BoNT/A was applied dropwise to the skin treated area followed by rubbing/massaging into the tissue with a polished glass rod. Following topical application, the treated TA muscles were collected and processed for SNAP25₁₉₇-positive staining by immunohistochemistry.

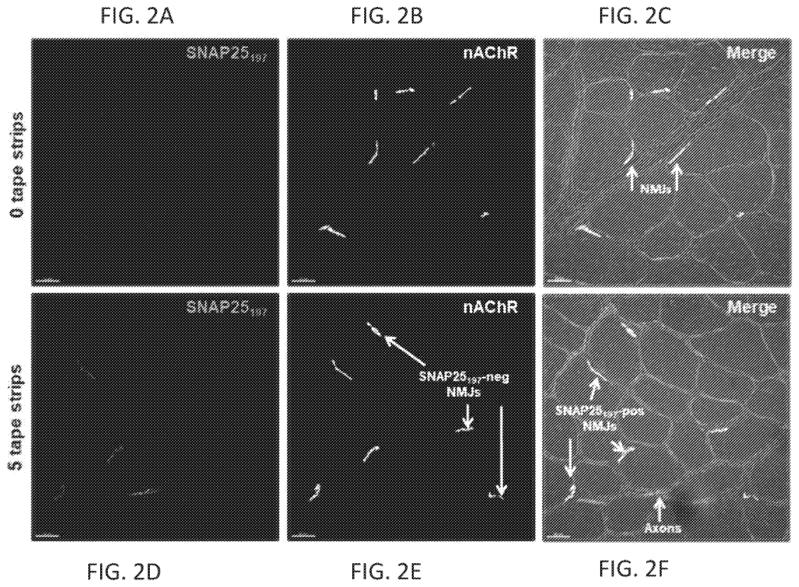

FIGS. 2A-2C show the stained tissue for the animals in the control group that were not subjected to tape stripping. FIGS. 2D-2F show the stained tissue for the animals treated with tape stripping before BoNT/A topical application. In the latter group, the TA muscle in each of the treated animals showed light SNAP25₁₉₇-positive staining in NMJs (FIG. 2E) and axons (FIG. 2D). The percent of SNAP25₁₉₇-labeled NMJs out of a total sampling of NMJs was estimated at ˜20%. No signal was observed in any TA muscles that did not undergo tape stripping to the overlying skin surface (FIGS. 2A-2C). These results demonstrate that tape stripping removes a portion of the stratum corneum, without causing damage to the underlying viable tissue, is sufficient to facilitate delivery of a biologic agent, such as a 150 kDa BoNT/A, to the dermis and to the superficial muscle layers.

Accordingly, a method for evaluating skin penetration of a topically applied biologic agent is provided. The method comprises providing a CD hairless rat and treating the skin in an indicated area with tape stripping. The indicated area, in one embodiment, is the skin overlying the TA muscle. After tape stripping, a formulation comprising a biologic agent is applied to the indicated (conditioned) area, and passive delivery of the agent into the skin is determined. In one embodiment, delivery into the skin comprises determining the depth of penetration of the agent using an in vitro or an in vivo technique. The in vitro technique can be, for example, microscopy or spectroscopy of a sample of the skin in the indicated area to which the formulation was applied where the skin can be stained or where the agent can be optically labeled prior to or after application to the skin. The in vivo technique can be, for example, the functional assay described herein or blood sampling for the presence (quantative or qualitative) of the agent in the blood of the animal.

The assay and methods described herein provide an approach to study delivery of a biologic agent to the dermis, hypodermis, and/or to superficial muscle from a topically applied formulation. The method comprises treating the skin to disrupt the stratum corneum, without disrupting the underlying dermis, of a CD hairless rat, or a haired rat that has been treated to remove the hair, and applying topically to the treated skin a formulation with the biologic agent. In one embodiment, the skin is skin of a CD hairless rat, or a haired rat that has been treated to remove the hair, and in another embodiment, the skin is skin on the leg of the CD hairless rat, or a haired rat that has been treated to remove the hair. The hairless rat skin has a skin thickness greater than typical rat skin. In the hairless rat, the leg skin surface to panniculus carnosus is about 1.2 mm. In humans, the distance from human face skin surface to cutaneous fat is about 2-3 mm. Barrier disruption facilitates delivery of the biologic agent through the non-intact hairless rat leg skin to the TA muscle at a distance of greater than ˜1.2 mm (the thickness of rat skin in this area).

In one embodiment, the biologic agent is delivered to the dermis, hypodermis, or superficial muscle solely and only by passive transport. Passive transport or diffusion relies on a concentration gradient between the drug at the outer surface and the inner surface of the skin. The diffusion rate is proportional to the gradient and is modulated by a molecule's size, hydrophobicity, hydrophilicity and other physiochemical properties as well as the area of the absorptive surface. In one embodiment, the biologic agent is delivered to the dermis or superficial muscle without any active transport. Active transport or delivery relies on, for example, ionization of the biologic agent, or other means to propel the agent into and through the skin. Active transport delivery systems include methods such as iontophoresis, sonophoresis, and thermal microporation.

The biologic agent contemplated for the methods described herein can have a molecular weight of greater than about 10 kDa, 25 kDa, 50 kDa, 75 kDa, 100 kDa, 125 kDa, 150 kDa, 175 kDa, 200 kDa, 250 kDa, 300 kDa, 350 kDa, 400 kDa, 500 kDa, 600 kDa, 700 kDa, 800 kDa, 900 kDa, 1,000 kDa, 1,500 kDa, 1,600 kDa, 2,000 kDa, 2,200 kDa, 2,500 kDa, or 3,000 kDa. The biologic agent contemplated for the methods described herein can have a molecular weight of greater than about 10 kDa, 25 kDa, 50 kDa, 75 kDa, 100 kDa, 125 kDa, 150 kDa, 175 kDa, 200 kDa, 250 kDa, 300 kDa, 350 kDa, 400 kDa, 500 kDa and less than about 3,000 kDa, 2,500 kDa, 2,200 kDa, 2,000 kDa, 1,600 kDa, 1,500 kDa, or 1,000 kDa.

In one embodiment, the biologic agent is a Clostridial derivative, such as a botulinum neurotoxins (BoNTs), such as, for example, BoNT/A, BoNT/B, etc. These toxins act on the nervous system by blocking the release of neurosecretory substances such as neurotransmitters. The action of BoNT is initiated by its binding to a receptor molecule on the cell surface, and then the toxin-receptor complex undergoes endocytosis. Once inside the cell, BoNT cleaves exocytotic specific proteins responsible for neurotransmitter docking and release from the cell known as the SNARE proteins (soluble N-ethylmaleimide-sensitive factor attachment protein receptor). The resulting transient chemodenervation has been utilized medically to block motor neurotransmission at the neuromuscular junction leading to a variety of therapeutic applications.

In some embodiments, the clostridial derivative includes a native, recombinant clostridial toxin, recombinant modified toxin, fragments thereof, TEMs, or combinations thereof. In some embodiments, the clostridial derivative is a botulinum toxin. In some embodiments, the botulinum toxin can be a botulinum toxin type A, type B, type C₁, type D, type E, type F, or type G, or any combination thereof. The botulinum neurotoxin can be a recombinantly made botulinum neurotoxins, such as botulinum toxins produced by E. coli. In alternative embodiments, the clostridial derivative is a TEM.

In some embodiments, the botulinum neurotoxin can be a modified neurotoxin, that is a botulinum neurotoxin which has at least one of its amino acids deleted, modified or replaced, as compared to a native toxin, or the modified botulinum neurotoxin can be a recombinant produced botulinum neurotoxin or a derivative or fragment thereof. In certain embodiments, the modified toxin has an altered cell targeting capability for a neuronal or non-neuronal cell of interest. This altered capability is achieved by replacing the naturally-occurring targeting domain of a botulinum toxin with a targeting domain showing a selective binding activity for a non-botulinum toxin receptor present in a non-botulinum toxin target cell. Such modifications to a targeting domain result in a modified toxin that is able to selectively bind to a non-botulinum toxin receptor (target receptor) present on a non-botulinum toxin target cell (re-targeted). A modified botulinum toxin with a targeting activity for a non-botulinum toxin target cell can bind to a receptor present on the non-botulinum toxin target cell, translocate into the cytoplasm, and exert its proteolytic effect on the SNARE complex of the target cell. In essence, a botulinum toxin light chain comprising an enzymatic domain is intracellularly delivered to any desired cell by selecting the appropriate targeting domain.

The Clostridial derivative, such as a botulinum toxin, for use according to the present methods can be stored in lyophilized, vacuum dried form in containers under vacuum pressure or as stable liquids. Prior to lyophilization the botulinum toxin can be combined with pharmaceutically acceptable excipients, stabilizers and/or carriers, such as, for example, albumin, or the like. In embodiments containing albumin, the albumin can be, for example, human serum albumin, or the like. The lyophilized material can be reconstituted with a suitable liquid such as, for example, saline, water, or the like to create a solution or composition containing the botulinum toxin to be administered to the patient.

In some embodiments, the clostridial derivative is provided in a controlled release system comprising a polymeric matrix encapsulating the clostridial derivative, wherein a fractional amount of the clostridial derivative is released from the polymeric matrix over a prolonged period of time in a controlled manner. Controlled release neurotoxin systems have been disclosed for example in U.S. Pat. Nos. 6,585,993; 6,585,993; 6,306,423 and 6,312,708, each of which is hereby incorporated by reference in its entirety.

In alternative embodiments, the Clostridial derivative is provided in an ointment, gel, cream, or emulsion suitable for topical administration.

The therapeutically effective amount of the Clostridial derivative, for example a botulinum toxin, administered according to the present method can vary according to the potency of the toxin and particular characteristics of the pain being treated, including its severity and other various patient variables including size, weight, age, and responsiveness to therapy. The potency of the toxin is expressed as a multiple of the LD₅₀ value for the mouse, one unit (U) of toxin being defined as being the equivalent amount of toxin that kills 50% of a group of 18 to 20 female Swiss-Webster mice, weighing about 20 grams each.

The therapeutically effective amount of the botulinum toxin can vary according to the potency of a particular botulinum toxin, as commercially available Botulinum toxin formulations do not have equivalent potency units. It has been reported that one Unit of BOTOX® (onabotulinumA), a botulinum toxin type A available from Allergan, Inc., has a potency Unit that is approximately equal to 3 to 5 Units of DYSPORT® (abobotulinumtoxinA), also a botulinum toxin type A available from Ipsen Pharmaceuticals. MYOBLOC®, a botulinum toxin type B available from Elan, has been reported to have a much lower potency Unit relative to BOTOX®. In some embodiments, the botulinum neurotoxin can be a pure toxin, devoid of complexing proteins, such as XEOMIN® (incobotulinumtoxinA). One unit of incobotulinumtoxinA has been reported to have potency approximately equivalent to one unit of onabotulinumtoxinA. Thus, the quantity of toxin administered and the frequency of its administration will be at the discretion of the physician responsible for the treatment and will be commensurate with questions of safety and the effects produced by a particular toxin formulation.

### EXAMPLES

The following non-limiting examples provide those of ordinary skill in the art with specific preferred methods within the scope of embodiments of the present methods and are not intended to limit the scope thereof.

### Example 1

### Effect of Tape Stripping on the Epidermal Barrier

D-SQUAME® tape strips (Clinical and Derm LLC (formerly Cuderm Corporation)) were applied with a finger tap to the leg of a CD hairless rat near the ankle. Mild thumb pressure was applied to the tape strip for 5 seconds. The tape was peeled from the skin unidirectionally, beginning at the foot. New tape strips were applied to the same area and the process was repeated for a total number of 5, 10, 20 or 30 strips. A control group received no tape stripping. The tape stripped area from each animal was collected along with the surrounding non-tape stripped skin, and preserved in 10% neutral buffered formalin. After an adequate fixation time, skin was routinely processed to paraffin blocks and glass slides, and then stained with hematoxylin and eosin for histologic evaluation. FIG. 1A is the histologic image of the skin from the leg of a CD hairless rat with no tape stripping. FIG. 1B is the image after tape stripping 5 times (FIG. 1B) and FIG. 1C is the image after tape stripping 10 times. All images are 200× magnification. The upper arrow in each image indicates the stratum corneum (or location of stratum corneum that has been removed in FIG. 1C image), and the lower arrow indicates the epidermis.

### Example 2

### Use of the Assay to Evaluate Skin Penetration of Functional Botulinum Neurotoxin

D-SQUAME® tape strips (Clinical and Derm LLC) were applied with forceps to the skin overlying the TA muscle of CD hairless rats and pressed for 5 seconds. The tape was peeled from the skin unidirectionally and a new tape strip was applied to the same application area. The process was repeated for a total number of 5 strips. A control group received no tape stripping.

50 μL of 7000 U/mL (350 U total) of 150 kDa BoNT/A was applied dropwise to the area of skin above the TA muscle followed by rubbing/massaging into the tissue with a polished glass rod. An intradermal injection of 10 U/kg 150 kDa BoNT/A was used as a positive control. Following topical application, the treated TA muscles were collected and process for SNAP25₁₉₇-positive staining by immunohistochemistry. Results are shown in FIGS. 2A-2C for the control group and in FIGS. 2D-2F for the group treated with tape stripping.

Many alterations and modifications may be made by those having ordinary skill in the art, without departing from the spirit and scope of the disclosure. Therefore, it must be understood that the described embodiments have been set forth only for the purposes of examples, and that the embodiments should not be taken as limiting the scope of the following claims. The following claims are, therefore, to be read to include not only the combination of elements which are literally set forth, but all equivalent elements for performing substantially the same function in substantially the same way to obtain substantially the same result. The claims are thus to be understood to include those that have been described above, those that are conceptually equivalent, and those that incorporate the ideas of the disclosure.

1. An in vivo assay to evaluate topical delivery of a biologic agent, comprising:
treating a skin area of a CD hairless rat with tape stripping to disrupt the stratum corneum in the skin area to define a treated skin area;
topically applying a formulation to the treated skin area, the formulation comprising the biologic agent and a pharmaceutically acceptable carrier; and
evaluating delivery of the biologic agent into the skin from the topically applied formulation.

2. The method of claim 1, wherein the treating comprises applying and removing a strip of tape between 2-9 times.

3. (canceled)

4. (canceled)

5. The assay of claim 2, wherein the treating comprises applying and removing a strip of tape using a different tape strip for each step of applying.

6. The assay of claim 2, wherein the step of applying comprises placing the tape strip to the skin applying mild thumb pressure for 5 seconds.

7. The assay of claim 1, wherein treating comprises treating with tape stripping using a strip of tape selected from the group of synthetic adhesive tapes sold under the brand names D-SQUAME®, Corneofix®, Blenderm™ and 3M-Scotch 845 Book Tape.

8. The assay of claim 1, wherein evaluating comprises evaluating in vitro.

9. The assay of claim 8, wherein the in vitro evaluating comprises a histologic evaluation of the treated skin area.

10. The assay of claim 1, wherein evaluating comprises evaluating in vivo.

11. The assay of claim 1, wherein the biologic agent is optically labeled and said evaluating comprises evaluating the treated skin area for the optical label.

12. The assay of claim 11, wherein evaluating for the optical label is via microscopy or spectroscopy.

13. The assay of claim 10, wherein evaluating comprises a functional assay of the rat.

14. The assay of claim 1, wherein evaluating comprises a determining a concentration of the biologic agent or a metabolite thereof in the blood of the rat.

15. The assay of claim 1, further comprising comparatively evaluating delivery of the biologic agent from the formulation applied to CD hairless rat skin not treated with tape stripping.

16. The assay of claim 1, wherein topically applying comprises topically applying a formulation comprising a biologic agent with a molecular weight of greater than about 100,000 Daltons.

17. The assay of claim 1, wherein topically applying comprises topically applying a formulation comprising a Clostridial derivative.

18. The assay of claim 17, wherein the Clostridial derivative is a botulinum toxin.

19. A method to evaluate skin penetration of a biologic agent from a topically applied formulation, comprising:
removing via tape stripping stratum corneum from a skin area of a CD hairless rat to define a conditioned skin area;
applying topically a formulation to the conditioned skin area, the formulation comprising a biologic agent and a pharmaceutically acceptable carrier; and
evaluating penetration of the biologic agent into the skin.

20. The method of claim 19, wherein the treating comprises applying and removing a strip of tape between 2-9 times.

21. (canceled)

22. (canceled)

23. The method of claim 19, wherein the treating comprises applying and removing a strip of tape using a different tape strip for each step of applying.

24. The method of claim 19, wherein the step of applying comprises placing the tape strip to the skin applying mild thumb pressure for 5 seconds.

25. The method of claim 19, wherein treating comprises treating with tape stripping using a strip of tape selected from the group of synthetic adhesive tapes sold under the brand names D-SQUAME®, Corneofix®, Blenderm™ and 3M-Scotch 845 Book Tape.

26. The method of claim 19, wherein evaluating comprises evaluating in vitro.

27. The method of claim 26, wherein the in vitro evaluating comprises a histologic evaluation of the treated skin area.

28. The method of claim 19, wherein evaluating comprises evaluating in vivo.

29. The method of claim 19, wherein the biologic agent is optically labeled and said evaluating comprises evaluating the treated skin area for the optical label.

30. The method of claim 29, wherein evaluating for the optical label is via microscopy or spectroscopy.

31. The method of claim 30, wherein evaluating comprises a functional assay of the rat.

32. The method of claim 19, wherein evaluating comprises a determining a concentration of the biologic agent or a metabolite thereof in the blood of the rat.

33. The method of claim 19, further comprising comparatively evaluating delivery of the biologic agent from the formulation applied to CD hairless rat skin not treated with tape stripping.

34. The method of claim 19, wherein topically applying comprises topically applying a formulation comprising a biologic agent with a molecular weight of greater than about 100,000 Daltons.

35. The method of claim 19, wherein topically applying comprises topically applying a formulation comprising a Clostridial derivative.

36. The method of claim 35, wherein the Clostridial derivative is a botulinum toxin.
